(19) United States
(12) Patent Application Publication
(10) Pub. No.: US 2005/0260150 A1
(43) Pub. Date: Nov. 24, 2005

(54) Polyol polyester-containing personal care products that do not contain low viscosity silicone fluids

(75) Inventor: Rocco Burgo, Mullica Hill, NJ (US)

Correspondence Address: FLASTER/GREENBERG P.C., 1810 CHAPEL AVENUE WEST, CHERRY HILL, NJ, 08002, US

(73) Assignee: INOLEX INVESTMENT CORP.

(21) Appl. No.: 10/849,991

(22) Filed: May 20, 2004

(51) Int. Cl. A61K 7/06; A61K 7/11

(52) U.S. Cl. 424/70.11

(57) ABSTRACT

The invention provides a personal care product that does not contain a lower viscosity silicone fluid; the personal care product includes at least one polyol polyester that is the esterification reaction product of a polyol having three to six carbons atoms and a carboxylic acid having three to six carbon atoms and a personal care formulation. Also provided are replacement compositions for use in personal care products that are at least one polyol polyester that is the esterification reaction product of a polyol having three to six carbons atoms and a carboxylic acid having three to six carbon atoms and having a kinematic viscosity of about 2 to about 20 centistokes at 25° C. and wherein the replacement composition does not contain a low viscosity silicone fluid. Related methods are also described.

### CROSS-REFERENCE TO RELATED APPLICATIONS

This application claims priority to U.S. Provisional Application Ser. No. 60/462,097, filed May 20, 2003, the contents of which are incorporated herein by reference.

Polyol polyester-containing personal care products that do not contain low viscosity silicone fluids.

### BACKGROUND OF THE INVENTION

Silicone fluids are widely used in toiletries, cosmetics, and other personal care products. Most frequently used are the lower viscosity silicone fluids, such as dimethicones and the cyclomethicones. Incorporated into a product, these silicone fluids provide a variety of benefits such as improved slip, reduction of tack, impartation of emolliency, and other modifications of the “feel” characteristics of the formulation. The lower viscosity silicone fluids have been considered to be particularly useful by personal care product formulators, as they tend to provide the aforementioned benefits without contributing to oiliness or greasiness, and give what may be considered to be a “dry” feel on skin. This attribute, as well as the others, is highly desirable in personal care products such as, for example, creams, lotions, antiperspirants, shaving products, and makeup products. Additional benefits are that the lower viscosity silicone fluids, particularly the tetramer (D4) and the pentamer (D5) cyclomethicones are excellent dispersing and spreading agents, are generally water white in color, low in odor, have some degree of volatility, and are resistant to chemical and oxidative attack.

There has been recent concern regarding the safety of the use of silicone fluids on the skin. For example, in light of potential links between D4 and D5 cyclomethicone and silicone degradation products to the development of autoimmune disorders in persons with silicone implants, such as silicone breast implants, these materials have recently come under higher scrutiny by the United States Food and Drug Administration. Although definitive links have not been confirmed, many personal care product formulators are taking steps to reduce and/or eliminate these ingredients from formulations. There is a need in the art to find alternative non-silicone fluids that provide the benefits of the lower viscosity silicone fluids without the real or perceived risks.

Attempts have been made to replace these lower viscosity silicone fluids in personal care products with commonly used esters, such as isopropyl laurate, isononyl isononanoate, and 2-ethylhexyl 2-ethylhexanoate. These esters provide some characteristics similar to the low viscosity silicone fluids; however, because they are monoesters, they tend to be slower spreading, oilier feeling, and may have a disagreeable odor when applied.

Thus, there remains an unmet need in the art for a compound that can provide in a personal care product the physical and chemical characteristics contributed by the lower viscosity silicone fluids.

### BRIEF SUMMARY OF THE INVENTION

The invention includes a personal care product comprising: (a) at least one polyol polyester that is the esterification reaction product of a polyol having three to six carbons atoms and a carboxylic acid having three to six carbon atoms; and (b) a personal care formulation, but which does not contain a low viscosity silicone fluid. Also provided is a personal care product comprising: (a) at least one polyol polyester that is the esterification reaction product of a polyol having three to six carbons atoms and a carboxylic acid having three to six carbon atoms; and (b) a personal care formulation, but which does not contain a low viscosity silicone fluid and wherein the personal care product has a performance characteristic similar to a personal care composition that contains a low viscosity silicone fluid.

The invention provides methods of preparing a replacement composition to replace a lower viscosity silicone fluid in a personal care composition, the replacement composition comprising at least one polyol polyester that is the esterification reaction product of a polyol having three to six carbons atoms and a carboxylic acid having three to six carbon atoms and having a kinematic viscosity of about 2 to about 20 centistokes at 25° C. and wherein the replacement composition does not contain a low viscosity silicone fluid.

A method of preparing a personal care product is described. The method comprises blending at least one polyol polyester that is the esterification reaction product of a polyol having three to six carbons atoms and a carboxylic acid having three to six carbon atoms with a personal care formulation, wherein the personal care product does not contain a lower viscosity silicone fluid.

### DETAILED DESCRIPTION OF THE INVENTION

The invention includes replacement compositions that may be incorporated into personal care products, personal care products containing the replacement compositions, and methods of preparing both the replacement composition and the personal care products. The replacement composition comprises certain polyol polyesters that compare favorably in performance properties (such as viscosity, spreading rate, and feel characteristics when incorporated into personal care products) with the characteristics of the lower viscosity silicone fluids. When practicing the invention, one may prepare personal care products that, to the end user, feel, apply, and perform like the lower viscosity silicone fluid-containing products.

By “personal care product” it is meant any composition that is used or marketed as a material to be applied to the skin, hair, or nails, and/or the stratum corneum of human or animal subjects for cosmetic, aesthetic and/or therapeutic effects, regardless of the delivery form of the composition, and may include phases or intermediate preparations ultimately formulated into personal care products. Such products can include grooming products, such as soaps, cleansers, shampoos, skin or hair conditioners, shaving creams, lotions, and gels; hair sprays, gels, tonics, mousses, pomades, and lacquers; antiperspirants and deodorants; skin lotions, creams, mousses, and ointments, and nail and cuticle care products, such as polishes and creams. Also included are color-imparting cosmetics (decorative cosmetics), such as lipsticks, mascaras, foundations, eye shadows, and other cosmetics that provide translucent or opaque color to the skin, hair or nail substrate.

By “lower viscosity silicone fluids” it is meant any silicone-containing fluids known or to be developed having a kinematic viscosity at 25° C. of about >20 cSt. measured using ASTM Official Method No. D-445-97 (1997), the contents of which are incorporated herein by reference.

The methods, replacement compositions, and personal care products of the invention each include one or more polyol polyesters derived from the esterification reaction of a polyol having about three to about six carbon atoms (C₃ to C₆) with a carboxylic acid having about three to about six carbon atoms (C₃ to C₆ linear or branched carboxylic acid). Polyols having one, two, three, four, five, six, seven, eight or nine carbon atoms may be preferred. The polyols may be branched or unbranched, substituted or unsubstituted. With respect to the selected carboxylic acids, one may also use those having one, two, three, four, five, six, seven, eight, or nine carbon atoms. The selected carboxylic acid(s) may be branched or unbranched, substituted or unsubstituted.

The characteristics desired in the end polyol polyester, such as flow properties like viscosity, viscosity temperature behavior, spreading rate, and skin feel properties, can be modified by a variance in selection and/or combination of the polyol(s) and carboxylic acid(s) used to prepare the polyol polyester. For example, to obtain low viscosity and low odor, diols having three to six carbon atoms may be preferred. Specific examples may include, without limitation, propylene glycol; 1,3-butylene glycol; 2-methyl-1,3-propanediol; diethylene glycol; dipropylene glycol; and neopentyl glycol.

Examples of preferred carboxylic acids for use may be, without limitation, propanoic acid, butyric acid, 2-methyl propanoic acid, 2-methyl butyric acid, isopentanoic acid, pentanoic acid, hexanoic acid, 2-ethyl butyric acid, and 2-methyl pentanoic acid.

The polyol polyester may be one having a structure represented by formula (I): 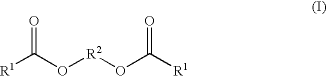
wherein R¹ is an aliphatic hydrocarbon of two to five carbon atoms and R² is an aliphatic hydrocarbon of three to six carbon atoms, and which contains zero to three ether linkages.

One exemplary polyol polyester for use in the replacement compositions of the invention may be neopentyl glycol dipentanoate. Neopentyl glycol dipentanoate (NDP) is a water white, odorless liquid with the viscosity of 5.1 centistokes at 25° C. NDP is a rapidly spreading liquid and provides a dry initial feel, and a soft silky, smooth, powdery after feel. It is particularly useful when used as an emollient in the formulation of creams, lotions, antiperspirants, color cosmetics, and hair care products in lieu of the lower viscosity silicone fluids.

Also included in the personal care products, replacement compositions, and methods of the invention are personal care formulations. By “personal care formulations,” it is meant any formulations known or to be developed in the art for personal care products. Exemplary formulations include those for soap (liquid, solid, foam, or mousse), cleansers, creams, lotions, ointments, suspensions, shampoos, deodorants, antiperspirants, conditioning products for hair, skin, and nails, and decorative cosmetics (i.e., those that impart color to the hair, skin, or nails upon application). The personal care formulations used in the invention may include organic or inorganic components; color active ingredients (e.g., pigments and dyes); therapeutic active ingredients (e.g., vitamins, alpha hydroxy acids, corticosteroids, amino acids, collagen, retinoids, antimicrobial compounds), sunscreens and/or UV absorbing compounds, reflective compounds, oils such as castor oil and olive oil; film formers, high viscosity oils, high molecular weight esters, antiperspirant active ingredients, glycol solutions, water, alcohols, emulsifiers, gellants, emollients, water, polymers, hydrocarbons, conditioning agents, and/or aliphatic esters.

Exemplary pigments and/or dyes include but are not limited to, titanium dioxide, iron oxides, zinc oxides, ultramarine blue, manganese violet, mica powder, barium lake, calcium lake, aluminum lake, FD&C yellow, FD&C green, FD&C blue, and FD&C red.

Suitable conditioning agents, especially for use in hair conditioning formulations, may include polyquaternium polymers, alkamidopropyldimethyl amines, alkyltrimonium chlorides, alkyltrimonium methyl sulfates, and ester quaternaries.

Exemplary high molecular weight esters include pentaerythritol, tetraisosterate (such as CRODAMOL® PTIS, Croda Corporation, Parsippany, N.J., United States of America) and dipentaerythritol hexa C₅-C₉ acid esters (such as LEXFEEL® 350, Inolex Chemical Company, Philadelphia, Pa., United States of America). In some cases, such as in a formulation for a personal care product that is a decorative cosmetic, it may be desirable to disperse the color active ingredients (e.g., opaque or translucent pigments or dyes) in these high molecular weight esters and/or high viscosity oils, such as a castor oil.

Typical film formers that can be used in the personal care formulation components of the invention include acrylate copolymers (such as AVALURE® AC120, Noveon Corporation, Cleveland, Ohio, United States of America), tricontanyl PDP (such as GANEX® WP-660, ISP Corporation, Wayne, N.J., United States of America), alpha-olefin (isopropyl maleate copolymer) (such as PROFORMA® V1608, New Faith Technologies, Sugarland, Tex., United States of America). In general, film formers will be included in the formulation when preparing a product intended to smooth out surface irregularities in the stratum corneum and to provide a stable immovable matrix for any color active ingredients to be included.

The personal care formulation may be an antiperspirant including inorganic salts and/or inorganic salt/glycine complexes. Examples of antiperspirant active ingredients include, but are not limited to, activated aluminum chlorohydrate (“AACH”), aluminum zirconium polychlorhydrex/glycine complex (“ZAG”) or activated ZAG (“AZAG”). The antiperspirant active ingredient may be provided to the formulation in any form, including a powder, an aqueous solution, or a glycol solution, such a propylene glycol, 1,3-butylene glycol, and dipropylene glycol.

Conventional antiperspirant gels are microemulsions made up of two immiscible phases in which the particle size of the internal phase is typically less than 100 nanometers. In conventional practice, the formulation of clear antiperspirant gels will include the combination of an active ingredient phase and a vehicle phase. The active ingredient phase will normally include one or more of the previously mentioned antiperspirant active ingredients, either dissolved or pre-dissolved in water, propylene glycol, other glycols, or mixtures thereof. The vehicle phase will typically include an emulsifier, gellant, and a mixture of oily ingredients that are meant to ease the spreading of the antiperspirant on to the skin, and to reduce the feeling of drag, greasiness, and/or tackiness. Common oily ingredients may be used in the vehicle phase of these non-silicone-containing personal care products such as hydrocarbons and aliphatic esters.

In the practice of the invention (regardless of the product or composition formed), the polyol polyester is formed by reacting a C₃-C₆ polyol as described above with a C₃-C₆ carboxylic acid to form a replacement composition that duplicates the spreading weight, viscosity, volatility, and/or skin feel of the lower viscosity silicone fluids. In this way, a replacement composition is provided. The reaction may be accomplished by any reaction protocol known or developed in the art. While not wishing to be bound by principle, it is hypothesized that the presence of at least two polar ester linkages in the finished polyol polyester provides the low volatility, low odor, and fast spreading rate on the skin. The specific carbon content of the polyols and carboxylic acids of C₃-C₆ provides the low viscosity necessary to match the lower viscosity silicone fluids, particularly the tetramer (D4) and pentamer (D5) cyclomethicone fluids. In addition, to match specific cyclomethicone fluids, such as D5, two or more polyol polyester(s) may be blended; however in the actual practice of cosmetic and toiletry product formulation this is not ordinarily necessary.

The personal care products of the invention can be prepared by mixing the replacement composition with a personal care formulation. Such blending may be accomplished by any means known in the art. Suitable means include mixing, blending, high sheer mixing, stirring, shaking or agitating.

Kinematic viscosity was tested at 25° C. using ASTM (American Society of Testing and Materials, West Conshohocken, Pa., United States of America) Official Method No. D-445-97 (1997), the contents of which are incorporated herein by reference. Color was measured using ASTM D-1209-97 (1997), acid value was determined using ASTM D-972-95 (1995) and hydroxyl number was determined using a modified version of AOCS (American Oil Chemists Society, Champaign, Ill., United States of America) Official Method No. Cd-13-60 (1997), the contents of which are incorporated herein by reference. Odor was determined using a subjective olfactory assessment.

The spreading characteristics of fluid used in personal care products are an effective predictor of “skin feel.” To test the spreading characteristics a four inch WHATMAN® filter paper was positioned horizontally over an eight ounce jar. Fifty microliters of each product were then pipetted onto the center of the filter paper. The spreading area of the liquid was then measured at intervals of one, three, and five minutes. Faster spreading products will have a higher spreading area at each time interval.

The names for the components given in the replacement composition and personal care product examples are those used by the Cosmetic, Toiletry, and Fragrance Association (“CTFA”), as set forth in the CTFA International Cosmetic Ingredient Dictionary and Handbook, 9^{th} ed. (2002).

### EXAMPLE 1

### Preparation of Replacement Composition to Replace Lower Viscosity Silicone Fluid in a Personal Care Product

Neopentyl glycol and n-pentanoic acid were charged to a stirred batch reactor in a molar ratio of 1:1.3 and heated with inert gas sparging to approximately 120° C. to 200° C. in the presence of a small quantity of a catalyst (0.1 wt % sulfonic acid). The hydroxyl value was monitored, and the reaction was stopped before the hydroxyl value reached three. The majority of the residual carboxylic acid was removed under reduced pressure. Remaining trace acid was neutralized with a sodium carbonate solution and the polyol polyester was then batch distilled. The distillate was then deodorized by steam stripping, and further purified with activated clay. It was then filtered. Table 1, below, shows the properties of the replacement composition obtained.

TABLE 1
Properties of the replacement composition.

| Property | Value |
|---|---|
| Color, APHA | <5 |
| Total acid number, mg KOH/g | 0.01 |
| Hydroxyl number, mg KOH/g | 0.5 |
| Odor | odorless |
| Viscosity at 25° C., cSt | 5.1 |
| Spreadibility (1 minute), cm | 3.08 |
| Spreadibility (3 minutes), cm | 3.48 |
| Spreadibility (5 minutes), cm | 3.85 |

### EXAMPLE 2

### Preparation of Replacement Composition to Replace Lower Viscosity Silicone Fluid in a Personal Care Product

Neopentyl glycol and 2-methylpropanoic acid (isobutyric acid) were charged to a stirred batch reactor in a molar ratio of 1:1.5 and heated with inert gas sparging to 180° C. in the presence of a small quantity of a catalyst (0.1 wt % sulfonic acid). The hydroxyl value was monitored, and the reaction was stopped when the hydroxyl value reached two. The majority of the residual carboxylic acid was removed under reduced pressure. The distillate was then deodorized by steam stripping, and further purified with activated clay. The distillate was then filtered. Table 2 shows the resultant properties of the replacement composition formulated in Example 2.

TABLE 2
Properties of Replacement Composition.

| Property | Value |
|---|---|
| Color, APHA | <5 |
| Total acid number, mg KOH/g | 0.001 |
| Hydroxyl number, mg KOH/g | 0.1 |
| Odor | slight |
| Viscosity at 25° C., cSt | 3.5 |
| Spreadibility (1 minute), cm | 3.18 |
| Spreadibility (3 minutes), cm | 3.63 |
| Spreadibility (5 minutes), cm | 3.88 |

### EXAMPLE 3

### Preparation of Replacement Composition to Replace Lower Viscosity Cyclomethicone in a Personal Care Product

Neopentyl glycol and 2-ethylbutyric acid were charged to a stirred batch reactor in a molar ratio of 1:1.5 and heated with inert gas sparging to 120° to 200° C. in the presence of a small quantity of a catalyst (0.1 wt % sulfonic acid). The hydroxyl value was monitored, and the reaction was stopped when the hydroxyl value reached three or lower. The majority of the residual carboxylic acid was removed under reduced pressure. Remaining trace acid was neutralized with a sodium carbonate solution, and the ester was then batch distilled. The distillate was then deodorized by steam stripping, and further purified with activated clay. It was then filtered. Table 3 shows the properties of the replacement composition, neopentyl glycol di-2-ethylbutyrate (“ND2EB”).

TABLE 3
Properties of Replacement Composition That Is ND2EB Ester.

| Property | Value |
|---|---|
| Color, APHA | 5 |
| Total acid number, mg KOH/g | 0.01 |
| Hydroxyl number, mg KOH/g | 2.4 |
| Odor | odorless |
| Viscosity at 25° C., cSt | 7.6 |
| Spreadibility (1 minute), cm | 3.00 |
| Spreadibility (3 minutes), cm | 3.43 |
| Spreadibility (5 minutes), cm | 3.63 |

### EXAMPLE 4

### Preparation of Replacement Composition to Replace Lower Viscosity Silicone Fluid in a Personal Care Product

Diethylene glycol and hexanoic acid were charged to a stirred batch reactor in a molar ratio of 1:1.5 and heated with inert gas sparging to 120° to 200° C. in the presence of a small quantity of catalysts. The hydroxyl value was monitored, and the reaction was stopped before the hydroxyl value reached three. The majority of the residual carboxylic acid was removed under reduced pressure. Remaining trace acid was neutralized with a sodium carbonate solution, and the replacement composition ester was then batch distilled. The distillate was then deodorized by steam stripping, and further purified with activated clay. It was then filtered. Table 4 illustrates the properties of the replacement composition containing diethylene glycol dihexanoate (“DEGDH”).

TABLE 4
Properties of the Replacement Composition:

| Property | Value |
|---|---|
| Color, APHA | 11 |
| Total acid number, mg KOH/g | 0.01 |
| Hydroxyl number, mg KOH/g | 2.2 |
| Odor | very slight |
| Viscosity at 25° C., cSt | 14.4 |
| Spreadibility (1 minute), cm | 2.98 |
| Spreadibility (3 minutes), cm | 3.43 |
| Spreadibility (5 minutes), cm | 3.68 |

### EXAMPLE 5

### Preparation of Replacement Composition to Replace Lower Viscosity Silicone Fluid in a Personal Care Product

To match the viscosity and skin feel of a D5 cyclomethicone fluid, the polyol polyester of Examples 2 and 1 (NDI and NDP) were blended in a ratio of 60:40 by weight. This replacement composition was evaluated, and Table 5 lists the properties of the replacement composition as compared to a D5 cyclomethicone fluid.

TABLE 5
Properties of Replacement Composition as Compared
to a D5 cyclomethicone Fluid

| Property | Replacement Composition | D5 Fluid |
|---|---|---|
| Viscosity at 25° C., cSt | 4.0 | 4.0 |
| Spreadibility (1 minute), cm | 3.12 | 2.9 |
| Spreadibility (3 minutes), cm | 3.63 | 3.4 |
| Spreadibility (5 minutes), cm | 3.93 | 3.6 |
| Odor | mild | slight |
| Initial skin feel | dry | dry |
| After skin feel | powdery | powdery |

### EXAMPLE 6

### Preparation of Replacement Composition to Replace Lower Viscosity Silicone Fluid in a Personal Care Product

Several replacement compositions were prepared in a manner similar to that described in the previous examples. The spreadibility properties of these replacement compositions were evaluated in comparison to the D4 and D5 cyclomethicone fluids. Table 6 shows the spreadibility data.

TABLE 6
Spreadibility Comparison.

| Spreadibility, cm | NDP | NDI | ND2EB | DEGDH | D4 | D5 |
|---|---|---|---|---|---|---|
| at 1 minute | 3.08 | 3.18 | 3.00 | 2.98 | 3.10 | 2.90 |
| at 3 minutes | 3.48 | 3.63 | 3.43 | 3.43 | 3.50 | 3.40 |
| at 5 minutes | 3.85 | 3.88 | 3.63 | 3.68 | 3.60 | 3.60 |

As can be seen from the data above, the spreadibilty at each of the time points of the replacement composition is similar to that exhibited by the D4 and D5 cyclomethicone fluids.

### EXAMPLE 7

### Preparation of a Silicone-Free Antiperspirant Stick Personal Care Product

A silicone-free antiperspirant stick personal care product was prepared using the replacement composition of the invention. Hydrogenated castor oil 2.5 g, stearyl alcohol 818.0 g and polypropylene glycol-14 butyl ether 9 g were combined in a vessel and warmed to approximately 80° to 85° C. under gentle agitation until a clear solution was obtained. Subsequently, the mixture was allowed to cool to approximately 70° C. The replacement composition of Example 5, 43 g, was warmed to approximately 70° C. and was added to the mixture. Maintaining the temperature at 70° C., talc 3 g, aluminum zirconium tetrachlorohydrex/glycine 24 g and amorphous fumed silica 0.5 g were added. The entire mixture was agitated until approximately uniformly mixed, then it was cooled to approximately 60° C. The resulting mixture was poured into canisters and allowed to cool to room temperature.

The resulting silicone-free antiperspirant stick personal care product exhibited the skin feel properties of an antiperspirant stick that contained a silicone component.

### EXAMPLE 8

### Preparation of a Silicone-Free Moisturizing Lotion Personal Care Product

A silicone-free moisturizing lotion personal care product was prepared. Deionized water 81.5 g, glycerin 3.0 g, and acrylates/C₁₀₋₂₀ alkyl acrylate cross polymer (PEMULEN® TR-1, available from Noveon Corporation, Cleveland, Ohio, United States of America), 0.2 g were combined in a vessel under agitation. When the PEMULEN® TR-1 was completely dispersed, the mixture was heated to approximately 70° to 75° C. Methylparaben 0.2 g and propylparaben 0.2 g were then added; the mixture was agitated until a uniform dispersion was obtained. Glyceryl stearate (and) polyethylene glycol-100 stearate (and) behenic acid (and) stearyl alcohol (and) behenamidopropyl PG dimonium chloride 1.5 g, glyceryl dilaurate 1.0 g, stearic acid 0.5 g and the replacement composition of Example 5, 10 g, were combined in a separate vessel and warmed to 70° to 75° C. under gentle agitation until a uniform dispersion was obtained. The contents of the first vessel were added to the second and the mixture was allowed to cool to approximately 60° C. The pH of the mixture was determined and was adjusted to approximately 6.0 with triethanolamine. The resulting mixture was then poured into containers and permitted to cool to room temperature.

### EXAMPLE 9

### Preparation of a Silicone-Free After-Bath Moisturizing Body Oil Personal Care Product

A silicone-free after-bath moisturizing body oil personal care product was prepared. First, trimethylolpropane tricaprylate/tricaprate 10 g, mineral oil 32 g, the replacement composition of Example 5, 56 g, and a fragrance 2 g were combined under agitation while being heated to approximately 45° C. The mixture is agitated under these conditions until a clear solution is obtained. The mixture is then poured into containers and permitted to cool to room temperature.

### EXAMPLE 10

### Preparation of a Silicone-Free Moisturizing Body Oil

A silicone-free moisturizing body oil was prepared. Jojoba oil 20 g, tocopheryl acetate 0.5 g, BHT 0.1 g, isopropylpalmitate 5.0 g, and the replacement composition of Example 1 74 g and a fragrance 0.4 g were combined under agitation in a vessel and heated to approximately 45° C. The mixture was agitated until a clear solution was obtained. It was then poured into containers and allowed to cool to room temperature.

### EXAMPLE 11

### Preparation of an Antiperspirant Stick Personal Care Product

An antiperspirant stick personal care product was prepared. Hydroxy stearic acid and the replacement composition of Example 3 were combined in a vessel and warmed to 83° C. under gentle agitation until a clear solution was obtained. Subsequently, the mixture was allowed to cool to approximately 70° C. Aluminum zirconium tetrachlorohydrex/glycine 24 g, talc 6 g, and silica 4 g were added. The mixture was agitated until uniform and then was cooled to approximately 60° C. The first mixture was then added to the second mixture, and mixed and combined thoroughly. The resulting mixture was poured into canisters and allowed to cool to room temperature.

### EXAMPLE 12

### Preparation of a Non-Silicone Containing Light Moisturizing Lotion Personal Care Product

A personal care product that is a light lotion that does not contain a silicone yet provides silicone-like aesthetics was prepared. Deionized water 77.52 g, acrylates/C₁₀₋₃₀ alkyl acrylate cross polymer 0.1 g, disodium EDTA 0.15 g, methylparaben 0.25 g, propylparaben 0.05 g were combined in a vessel and warmed to approximately 73° C. under vigorous agitation until a clear, uniform solution was obtained. Glyceryl stearate (and) polyethylene glycol-100 stearate 3.0 g, glyceryl stearate 2.0 g, cetearyl alcohol 1.5 g and DEG/C6 ester 15 g were combined in a separate vessel and warmed to approximately 73° C. until a clear solution was obtained.

The second solution was added to the first solution under high sheer mixing. The high sheer mixer is stopped and a 10% aqueous solution of sodium hydroxide 0.43 g was added under gentle agitation. The heating was stopped and the solution was allowed to cool to room temperature under gentle agitation. The resulting emulsion was poured into canisters.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A personal care product comprising:
(a) at least one polyol polyester that is the esterification reaction product of a polyol having three to six carbons atoms and a carboxylic acid having three to six carbon atoms;
(b) a personal care formulation, but which does not contain a low viscosity silicone fluid.

2. The product of claim 1, wherein the at least one polyol polyester independently contains at least one to three ether linkages.

3. The product of claim 1, wherein the polyol is independently selected from the group consisting of propylene glycol; 1,3-butylene glycol; 2-methyl-1,3-propanediol; diethylene glycol; neopentyl glycol; triethylene glycol; and dipropylene glycol.

4. The product of claim 1, wherein the carboxylic acid is selected from the group consisting of propanoic acid, butyric acid, 2-methyl propanoic acid, 2-methyl butyric acid, isopentanoic acid, pentanoic acid, hexanoic acid, 2,ethylbutyric acid, and 2-methyl pentanoic acid.

5. The product of claim 1, wherein the at least one polyol polyester has a structure represented by formula (I): 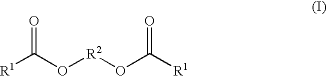
wherein R¹ is an aliphatic hydrocarbon of about two to about five carbon atoms and R² is an aliphatic hydrocarbon of about three to about six carbon atoms, and which contains zero to three ether linkages:

6. The product of claim 1, wherein the personal care formulation is selected from the group consisting of an antiperspirant personal care formulation, a hair conditioning personal care formulation, and a decorative cosmetic formulation.

7. A personal care product comprising:
(a) at least one polyol polyester that is the esterification reaction product of a polyol having three to six carbons atoms and a carboxylic acid having three to six carbon atoms;
(b) a personal care formulation,
but which does not contain a low viscosity silicone fluid and wherein the personal care product has a performance characteristic similar to a personal care composition that contains a low viscosity silicone fluid.

8. A replacement composition to replace a lower viscosity silicone fluid in a personal care composition, the replacement composition comprising at least one polyol polyester that is the esterification reaction product of a polyol having three to six carbons atoms and a carboxylic acid having three to six carbon atoms and having a kinematic viscosity of about 2 to about 20 centistokes at 25° C. and wherein the replacement composition does not contain a low viscosity silicone fluid.

9. A method of preparing a personal care product comprising blending at least one polyol polyester that is the esterification reaction product of a polyol having three to six carbons atoms and a carboxylic acid having three to six carbon atoms with a personal care formulation, wherein the personal care product does not contain a lower viscosity silicone fluid.

10. The method of claim 9, wherein the at least one polyol polyester independently contains at least one to three ether linkages.

11. The method of claim 9, wherein the polyol is independently selected from the group consisting of propylene glycol; 1,3-butylene glycol; 2-methyl-1,3-propanediol; diethylene glycol; neopentyl glycol; triethylene glycol; and dipropylene glycol.

12. The method of claim 9, wherein the carboxylic acid is selected from the group consisting of propanoic acid, butyric acid, 2-methyl propanoic acid, 2-methyl butyric acid, isopentanoic acid, pentanoic acid, hexanoic acid, 2,ethylbutyric acid, and 2-methyl pentanoic acid.

13. The method of claim 9, wherein the at least one polyol polyester has a structure represented by formula (I): 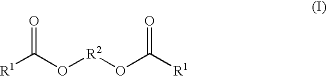
wherein R¹ is an aliphatic hydrocarbon of about two to about five carbon atoms and R² is an aliphatic hydrocarbon of about three to about six carbon atoms, and which contains zero to three ether linkages.

14. The method of claim 9, wherein the personal care formulation is selected from the group consisting of an antiperspirant personal care formulation, a hair conditioning personal care formulation, and a decorative cosmetic formulation.
